(19) United States
(12) Patent Application Publication
(10) Pub. No.: US 2019/0022426 A1
(43) Pub. Date: Jan. 24, 2019

(54) Targeted Monitoring of Nervous Tissue Activity

(71) Applicant: UNIVERSITY OF WASHINGTON, Seattle, WA (US)

(72) Inventors: Pierre D. Mourad, Seattle, WA (US); Felix Darvas, Seattle, WA (US)

(73) Assignee: UNIVERSITY OF WASHINGTON, Seattle, WA (US)

(21) Appl. No.: 16/077,167

(22) PCT Filed: Feb. 10, 2017
(86) PCT No.: PCT/US17/17521

§ 371 Date: Aug. 10, 2018

Related U.S. Application Data
(60) Provisional application No. 62/294,207, filed on Feb. 11, 2016.

(51) Int. Cl. A61N 7/00; A61B 5/0482

(52) U.S. Cl. CPC A61N 7/00; A61B 5/0478; A61N 2007/0026; A61B 5/0482

(57) ABSTRACT

An example method includes applying ultrasound waves to a particular portion of a nervous tissue. The ultrasound waves are pulsed at a pulse repetition frequency. The method further includes detecting an electrical signal originating from at least the particular portion of the nervous tissue. The method further includes extracting a component of the electrical signal that oscillates at an oscillation frequency that is equal to (a) the pulse repetition frequency, (b) a subharmonic frequency of the pulse repetition frequency, or (c) a harmonic frequency of the pulse repetition frequency. The method further includes processing the extracted component to obtain one or more components that oscillate at respective frequencies that are unequal to the pulse repetition frequency. Systems and computer readable media related to the example method are disclosed herein as well.

### CROSS-REFERENCE TO RELATED APPLICATION

This application claims the benefit of U.S. Provisional Patent Application No. 62/294,207, filed on Feb. 11, 2016, the contents of which are incorporated herein by reference in their entirety.

### BACKGROUND

Unless otherwise indicated herein, the materials described in this section are not prior art to the claims in this application and are not admitted to be prior art by inclusion in this section.

Current techniques for monitoring the activity of nervous tissue (e.g., brain tissue) include electroencephalography (EEG) and electrocorticography (ECoG). EEG typically involves placing electrodes on a subject's scalp and using the electrodes to measure voltage fluctuations resulting from neural activity within the brain. ECoG typically involves placing electrodes on a surgically exposed surface of a subject's brain to measure voltage fluctuations resulting from neural activity within the brain.

The voltage fluctuations detected via EEG or ECoG are generally not attributable to particular areas of the brain for a number of reasons. For example, signals originating from superficial areas of the brain may arrive at the electrodes simultaneously with signals originating from internal regions of the brain. Additionally, such signals may suffer from motion-induced noise artifacts. Also, when signals that originate from internal areas of the brain are detected, they are generally much weaker than signals that originate from superficial areas of the brain due to attenuation that occurs as the signals travel through brain tissue to the electrode. ECoG mitigates these issues somewhat, but comes at the cost of increased invasiveness to the subject.

### SUMMARY

In one example, a method includes applying ultrasound waves to a particular portion of a nervous tissue. The ultrasound waves are pulsed at a pulse repetition frequency. The method further includes detecting an electrical signal originating from at least the particular portion of the nervous tissue. The method further includes extracting a component of the electrical signal that oscillates at an oscillation frequency that is equal to (a) the pulse repetition frequency, (b) a subharmonic frequency of the pulse repetition frequency, or (c) a harmonic frequency of the pulse repetition frequency. The method further includes processing the extracted component to obtain one or more components that oscillate at respective frequencies that are unequal to the pulse repetition frequency.

In another example, a computer readable medium stores instructions that, when executed by a system, cause the system to perform functions. The functions include applying ultrasound waves to a particular portion of a nervous tissue. The ultrasound waves are pulsed at a pulse repetition frequency. The functions further include detecting an electrical signal originating from at least the particular portion of the nervous tissue. The functions further include extracting a component of the electrical signal that oscillates at an oscillation frequency that is equal to (a) the pulse repetition frequency, (b) a subharmonic frequency of the pulse repetition frequency, or (c) a harmonic frequency of the pulse repetition frequency. The functions further include processing the extracted component to obtain one or more components that oscillate at respective frequencies that are unequal to the pulse repetition frequency.

In yet another example, a system includes one or more processors, an ultrasound transducer, one or more sensors, and a computer readable medium. The computer readable medium stores instructions that, when executed by the one or more processors, cause the system to perform functions. The functions include applying ultrasound waves, via the ultrasound transducer, to a particular portion of a nervous tissue. The ultrasound waves are pulsed at a pulse repetition frequency. The functions further include detecting, via the one or more sensors, an electrical signal originating from at least the particular portion of the nervous tissue. The functions further include extracting a component of the electrical signal that oscillates at an oscillation frequency that is equal to (a) the pulse repetition frequency, (b) a subharmonic frequency of the pulse repetition frequency, or (c) a harmonic frequency of the pulse repetition frequency. The functions further include processing the extracted component to obtain one or more components that oscillate at respective frequencies that are unequal to the pulse repetition frequency.

When the term “substantially” or “about” is used herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. In some examples disclosed herein, “substantially” or “about” means within +/−5% of the recited value.

These, as well as other aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate the invention by way of example only and, as such, that numerous variations are possible.

### BRIEF DESCRIPTION OF THE DRAWINGS

FIG. 1 is a schematic diagram of a system, according to an example embodiment.

FIG. 2 is a block diagram of a method, according to an example embodiment.

FIG. 3 depicts application of ultrasound waves to nervous tissue and detection of electrical signals originating from nervous tissue, according to an example embodiment.

FIG. 4 depicts application of ultrasound waves to nervous tissue and detection of electrical signals originating from nervous tissue, according to an example embodiment.

FIG. 5 is a schematic diagram of ultrasound waves applied to nervous tissue, according to an example embodiment.

FIG. 6 depicts EEG amplitude response with respect to differing target characteristics, according to an example embodiment.

FIG. 7 depicts EEG amplitude response of four rat subjects, according to an example embodiment.

FIG. 8 depicts EEG frequency response with respect to differing target characteristics, according to an example embodiment.

### DETAILED DESCRIPTION

As discussed above, current techniques for monitoring nervous tissue activity via EEG or ECoG have disadvantages such as invasiveness and/or inability to attribute detected neural activity to a particular portion of the nervous tissue being monitored. Approaches for alleviating these issues are discussed herein.

For instance, an ultrasound transducer may apply pulsed focused ultrasound (pFU) waves that are selectively focused upon a particular portion of nervous tissue, such as brain tissue. The pFU may be sinusoidal, have a carrier frequency of 2 MHz, a pulse duration of 200 μs, a spatial peak temporal average intensity (I_{SPTA}) of 1.4 W/cm², and/or a pulse repetition frequency (PRF) of 1.05 kHz, but other examples are possible. Additionally, the pFU may be cycled on and off at a duty cycle of 50% over a period of 2 seconds. Other examples are possible as well.

In some examples, the particular portion of nervous tissue may be an internal region of a subject's brain tissue that is of particular interest. The relatively high frequency pFU waves may selectively “tag” or become superimposed upon the naturally occurring lower frequency signals that originate from the particular portion of the brain, such that those signals are recognizable as originating from the particular portion of the brain. That is, in addition to the particular portion of the brain tissue exhibiting neural activity in the form of low frequency electrical oscillations (e.g., 3 to 1000 Hz), the particular portion of the brain tissue may, in response to the pFU, also exhibit neural/electrical activity in the form of high frequency oscillations. The high frequency oscillations may be equal to the pulse repetition frequency (PRF) of the pFU (e.g., 1.05 kHz), equal to harmonic frequencies (e.g., integer multiples) of the PRF, or equal to subharmonic frequencies of the PRF (e.g., f=PRF/n, where ‘n’ is an integer).

It may be advantageous (e.g., less invasive) to place electrodes upon the scalp of the subject, although the electrodes may be placed on an exposed cranium or on exposed brain tissue as well. The electrodes may detect signals originating from various regions of the brain. For example, the electrodes may detect signals originating from the particular portion of the brain and simultaneously detect signals originating from other regions of the brain. Such signals may be superimposed on each other such that signal processing may be useful to distinguish the various signals.

For example, a band pass filter having a center frequency equal to or substantially equal to the PRF (e.g., 1.05 kHz) may be used to extract, from among all signal components detected by the electrodes, one or more components that originate from the particular portion of the brain. That is, by applying pFU only to the particular portion of the brain, one can be confident that any extracted signal component oscillating at the PRF is representative of the particular portion of the brain and not other regions of the brain.

After the high-frequency component of the signal corresponding to the particular portion of the brain is extracted, hardware or software processing or demodulation (e.g., amplitude demodulation) may be performed to reconstruct the low frequency (e.g., naturally occurring) signals originating from the particular portion of the brain. Software processing or demodulation may involve mathematical processing of the digitized signals, whereas hardware solutions may involve a diode rectifier envelope detector, a product detector, and/or synchronous detection. This may yield a signal representing naturally occurring neural activity of the particular portion of the brain.

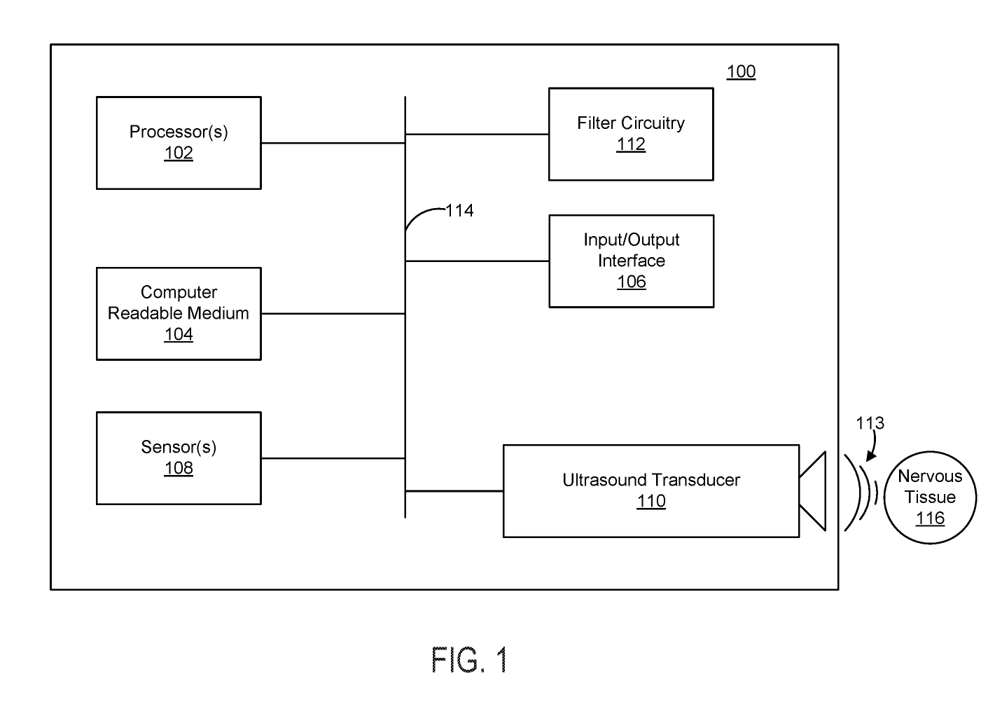

Referring now to the Figures, FIG. 1 illustrates an example system 100 configured to “tag” a particular portion of nervous tissue 116 to facilitate monitoring of neural activity within the particular portion of the nervous tissue 116. The system 100 may include one or more processors 102, a computer readable medium 104, an input/output interface 106, one or more sensors 108, an ultrasound transducer 110, and filter circuitry 112, any or all of which may be communicatively coupled to each other via a system bus or another connection mechanism 114.

The processor 102 may include a general purpose processor and/or a special purpose processor and may be configured to execute program instructions stored within the computer readable medium 104. In some examples, the processor 102 may be a multi-core processor comprised of one or more processing units configured to coordinate to execute instructions stored within computer readable medium 104. In one example, the processor 102, by executing program instructions stored within computer readable medium 104, may provide ultrasound parameters to the ultrasound transducer 110 for generation and/or directional focusing of pFU waves. In another example, the processor 102 may provide pFU parameters that are received via the input/output interface 106 to the ultrasound transducer 110. Such ultrasound parameters may include intensity, pulse duration, PRF, carrier frequency, and/or duty cycle, for example.

Computer readable medium 104 may include one or more volatile, non-volatile, removable, and/or non-removable storage components. Computer readable medium 104 may be a magnetic, optical, or flash storage medium, and may be integrated in whole or in part with the processor 102 or other portions of the system 100. Further, the computer readable medium 104 may be a non-transitory computer-readable storage medium, having stored thereon program instructions that, when executed by the processor 102, cause the system 100 to perform any functions described in this disclosure. Such program instructions may be part of a software application that can be executed in response to inputs received from the input/output interface 106, for instance. The computer readable medium 104 may also store other types of information or data, such as those types described throughout this disclosure.

The input/output interface 106 may enable interaction with a user of the system 100, if applicable. The input/output interface 106 may include input components such as dials, buttons, a keyboard, a mouse, a keypad, or a touch-sensitive panel, and output components such as a display screen (which, for example, may be combined with a touch-sensitive panel), a sound speaker, and a haptic feedback system. In one example, the input/output interface 106 may receive input indicating (i) various parameters defining a pFU wave to be generated by the ultrasound transducer 110 and/or (ii) various parameters for sequentially directing the focal point of the pFU wave upon various portions of the nervous tissue 116.

In some examples, the input/output interface 106 may include a display screen for displaying images of the nervous tissue 116 or other sensory data collected by the sensors 108. Properly determining a trajectory for ablating the nervous tissue 116 will generally require characterizing the size, shape, location, and/or consistency of the nervous tissue 116. The display screen may display images of the nervous tissue 116 that are captured by the sensors 108. The displayed images of the nervous tissue 116 may be used prior to determine a suitable trajectory, or could be used in a real-time manner by monitoring progress of the nervous tissue 116 and adjusting the trajectory accordingly.

The sensors 108 may include electrodes or other means for detecting electrical signals (e.g., voltage fluctuations) that originate from the nervous tissue 116. In an example where the nervous tissue 116 is part of a subject's brain, the sensors 108 may be applied to the subject's scalp, surgically exposed cranium, or surgically exposed brain surface. In an example where the nervous tissue 116 is part of a subject's spinal cord, the sensors 108 may be applied similarly in the vicinity of the spinal cord.

The ultrasound transducer 110 may include a signal generator configured to receive data from the processor 102 or input/output interface 106 that is representative of parameters for the pFU wave 113. For instance, the processor 102 may send, to the ultrasound transducer 110, data representative of input received via the input/output interface 106. Such data received by the ultrasound transducer 110 may indicate various pFU parameters such as operating power of the ultrasound transducer 110, power density of the pFU wave 113, carrier frequency of the pFU wave 113, pulse duration of the pFU wave 113, duty cycle of the pFU wave 113, and a number of pFU pulses to be generated. The received data may also indicate a target portion of the nervous tissue 116 upon which the focal point of the pFU wave 113 should be directed upon. In other examples, the path of the pFU wave 113 may be manually and/or mechanically directed. In some examples, the ultrasound transducer 110 may include a signal amplifier used to generate the pFU wave 113 at a desired power.

The ultrasound transducer 110 may include one or more piezoelectric transducer elements configured to generate pFU waves in response to receiving respective control signals representing pFU parameters. For example, the ultrasound transducer 110 may include a phased array of transducer elements configured to electronically focus or steer a generated pFU wave upon various portions of the nervous tissue 116 via constructive and/or destructive wave interference. Each transducer element of the ultrasound transducer 110 may receive its own independent control signal. The ultrasound transducer 110 may also include one or more of (i) a lens, (ii) one or more transducers having a radius of curvature at the focal point of the pFU wave, and (iii) a phased array of transducers.

Filter circuitry 112 may include one or more electrical components, such as diodes, capacitors, or resistors that are configured to perform filter operations and or other processing of detected electrical signals. In some examples, electrical signals may be processed via software means, that is, via the processor 102 and the computer readable medium 104.

The nervous tissue 116 may include brain or spinal cord tissue of a living or dead human or animal subject.

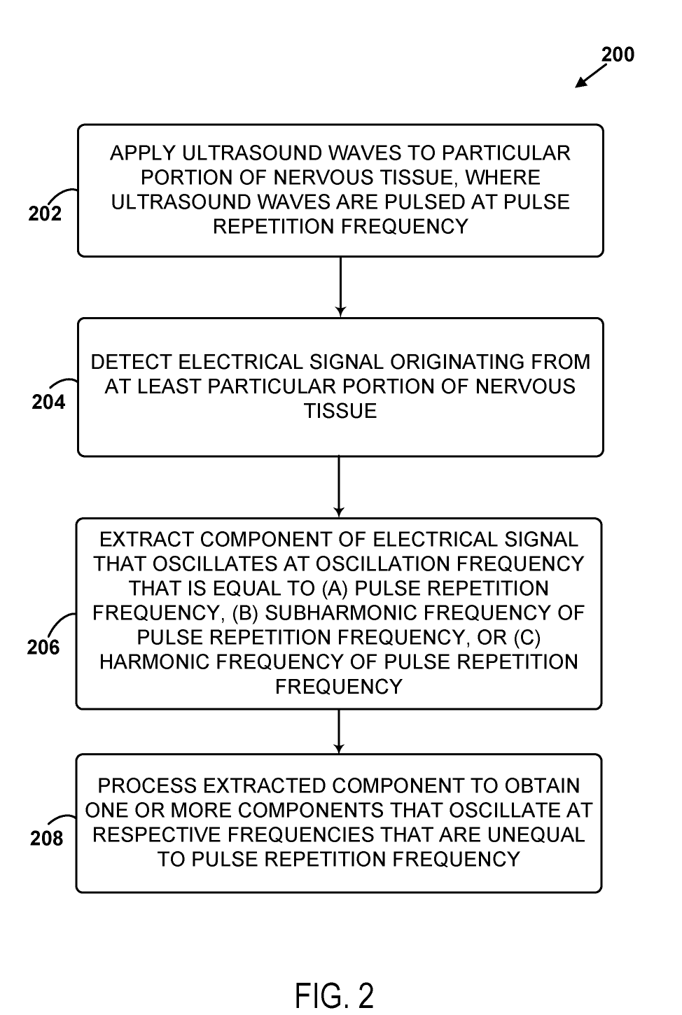

FIG. 2 is a block diagram of a method 200 for monitoring the electrical/neural activity of nervous tissue.

At block 202, the method 200 includes applying ultrasound waves to a particular portion (e.g., a portion of interest) of the nervous tissue. This may serve to “tag” the particular portion of the nervous tissue, that is, induce a disturbance within the particular portion of the nervous tissue that is recognizable as being caused by application of the ultrasound waves.

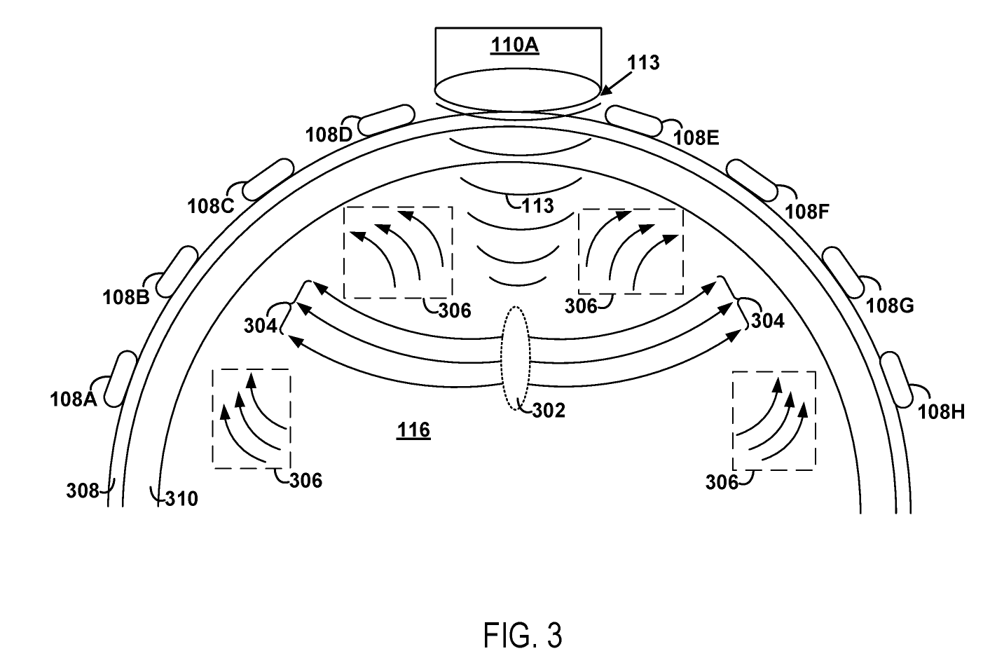

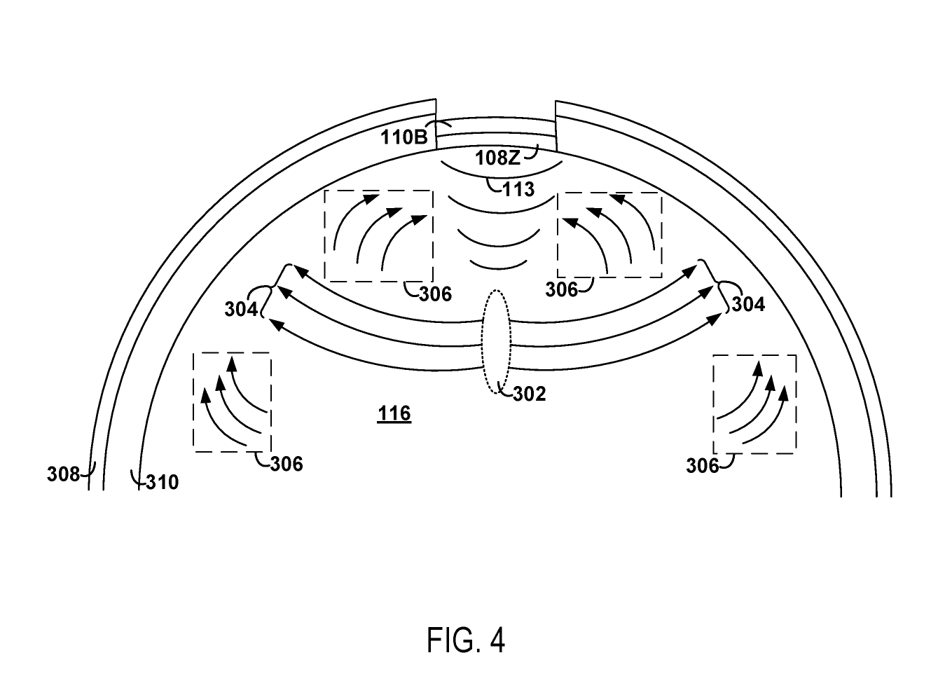

As shown in FIGS. 3 and 4, an ultrasound transducer may apply the ultrasound waves 113 that are focused upon a particular portion 302 of the nervous tissue 116. In these examples, the portion 302 may be surrounded by other portions of the nervous tissue 116 (e.g., brain tissue), but other examples are possible.

The ultrasound transducer 110 of FIG. 1 may take the form of an ultrasound transducer 110A as shown in FIG. 3. In this example, the ultrasound transducer 110A applies the ultrasound waves 113 from a position that is external to a subject's scalp 308. In another example, the ultrasound transducer 110A may apply the ultrasound waves 113 while positioned against an exposed cranium, or against exposed brain tissue.

In another example, the ultrasound transducer 110 of FIG. 1 may take the form of an ultrasound transducer array 110B as shown in FIG. 4. In this example, the ultrasound transducer array 110B may be surgically implanted within a hole in the subject's cranium 310. In another example, an ultrasound transducer array might be implanted underneath the scalp, but external to the subject's cranium. In yet another example, the ultrasound transducer array 110B might be implanted upon exposed brain tissue. Other examples are possible.

The ultrasound waves 113 may be pulsed at a pulse repetition frequency (PRF) that range anywhere from 1 Hz to 20 MHz. In a particular example, the pulse repetition frequency is equal to 1.05 kHz.

The ultrasound waves 113 may have a carrier frequency ranging anywhere from 20 kHz to 200 MHz. In a particular example, the carrier frequency may be 2 MHz.

The ultrasound waves 113 may have a pulse duration within a range of 1-500 μs. In a particular example, the pulse duration may be equal to 200 μs.

The ultrasound waves may have a spatial peak temporal average intensity (I_{SPTA}) within a range of 0.01-20 W/cm² as measured within the nervous tissue 116.

At block 204, the method 200 includes detecting an electrical signal originating from at least the particular portion of the nervous tissue. The sensors 108 of FIG. 1 may take the form of sensors 108A, 108B, 108C, 108D, 108E, 108F, 108G, and 108H as shown in FIG. 3. The sensors 108A-H may detect one or more of the electrical signals 304 and 306 and may take the form of electrodes adhesively or otherwise attached to the subject's scalp 308.

In the example of FIG. 4, the sensors 108 of FIG. 1 may take the form of a sensor array 108Z that is implanted within a surgically created hole in the scalp 308 and/or the cranium 310. As such, the sensor array 108Z may detect one or more of the electrical signals 304 and 306.

In either case, one or more sensors may detect one or more of the signals 304 and 306 as a composite signal representing a superposition of the signals 304 and 306 in a manner similar to known EEG or ECoG techniques. The signals 304 may originate from the portion 302 of the nervous tissue 116, whereas the signals 306 may originate from other portions of the nervous tissue 116. The signals 304 and 306 may represent electrical/neural activity of the portions of the nervous tissue 116 from which the signals 304 and 306 respectively originate. The electrical signals 304 may include artifacts of the ultrasound waves 113 that are focused upon the portion 302 of the nervous tissue 116. Otherwise the electrical signals 304 may generally reflect naturally occurring electrical/neural activity within the portion 302.

At block 206, the method 200 includes extracting a component of the electrical signal that oscillates at an oscillation frequency that is equal to (a) the pulse repetition frequency (PRF), (b) a subharmonic frequency of the pulse repetition frequency, or (c) a harmonic frequency of the pulse repetition frequency. For example, the sensors 108A-H or the sensor array 108Z may detect a composite signal representing a superposition of the signals 304 and 306. The system 100 may selectively extract a component of the detected composite signal that contains artifacts of the ultrasound waves 113 (e.g., frequency components equal to the PRF, harmonics of the PRF, or subharmonics of the PRF). As such, it can be inferred that the extracted component originates only from the portion 302 of the nervous tissue 116 because the ultrasound waves 113 are focused upon the portion 302 and because frequency components equal to the PRF or that are harmonics/subharmonics of the PRF will generally not occur naturally within the nervous tissue 116.

The system 100 may extract the signal component corresponding to the portion 302 by using a high pass filter, a bandpass filter, or other hardware or software means. For example, the system 100 may use a low pass filter with a corner frequency slightly lower than or equal to the PRF of the ultrasound waves 113, or a bandpass filter (e.g., a 4^{th} order Butterworth filter) having a center frequency approximately equal to the PRF.

As such, the extracted signal component may have one or more frequency components that are equal to the PRF of the ultrasound waves 113, equal to harmonic frequencies corresponding to the PRF (e.g., integer multiples of the PRF), or other frequencies that are greater than the PRF.

At block 208, the method 200 includes processing the extracted component to obtain one or more components that oscillate at respective frequencies that are unequal to the pulse repetition frequency. In various examples, the respective frequencies of the one or more obtained components might not be equal to subharmonic/harmonic frequencies of the PRF either. For instance, amplitude demodulation or other processing may be used to reconstruct a signal envelope that is “carried” by the higher frequency (e.g., 1.05 kHz) carrier wave of the detected composite signal. The obtained envelope may include frequency components ranging anywhere from 3 Hz to 1000 Hz. Demodulation or other processing techniques may employ a diode rectifier envelope detector, a product detector, and/or synchronous detection. Other examples are possible. As a result of this process, the one or more signal components obtained via demodulation or other processing are generally representative of naturally occurring electrical/neural activity that can be inferred to have occurred within the portion 302 of the nervous tissue 116.

The above techniques may be used to diagnose or treat subjects having a nervous system disorder or exhibiting symptoms of a nervous symptom disorder such as epilepsy, traumatic brain injury, or depression. Other examples are possible. Information obtained via these methods may be used to guide targeting and power parameters for therapeutic ultrasound, for example.

It may be useful to enhance or suppress, via therapeutic ultrasound, certain neurological activity that is detected within a subject. For instance, certain portions of the brain are known to be associated with various nervous system disorders and/or brain functions. The above methods can be used to determine whether such portions of the brain are functioning normally, and if not, to enhance beneficial brain activity or suppress harmful or anomalous brain activity in those portions of the brain.

It is also known that certain “primary” portions of the brain control the activity of other “secondary” portions of the brain, so the above methods may be used to indirectly alter the activity of “secondary” portions of the brain by altering the activity of the “primary” portions of the brain.

The following includes description of experimental results of methods similar to those described above being performed upon living and dead rat brain tissue, as well as alginate.

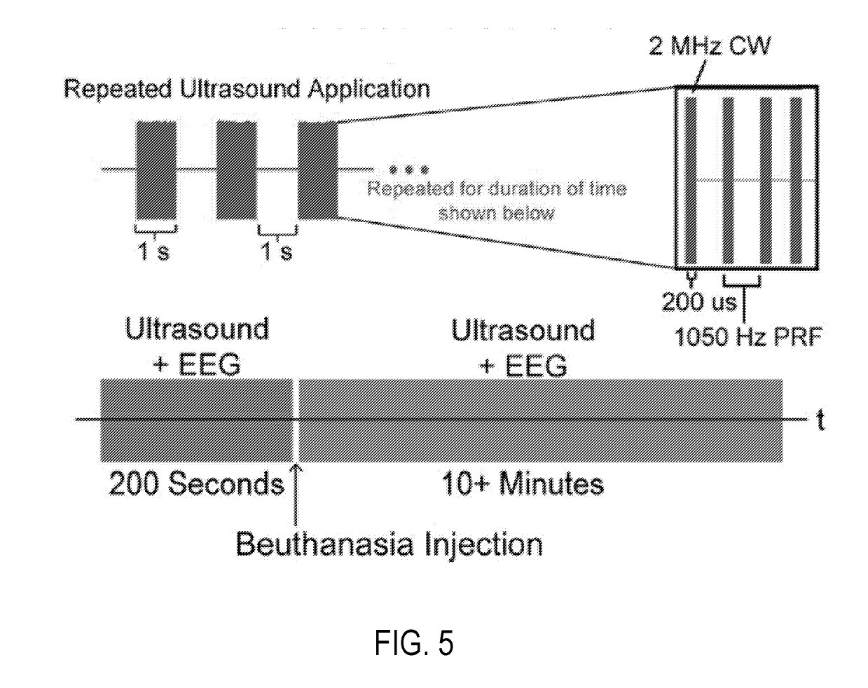

FIG. 5 is a schematic diagram of example ultrasound waves applied to brain tissue of a rat subject. The ultrasound waves were defined by pulses of 200 μs, a carrier frequency of 2 MHz, and a pulse repetition frequency (PRF) of 1050 Hz. This pattern was applied for one second, followed by a one second period with no ultrasound applied. This on/off period lasted for 100 repetitions (200 seconds) during which EEG was continuously recorded. This was immediately followed by injecting the rat with Beuthanasia (for euthanasia) lasting approximately 30 seconds followed immediately by continued ultrasound application and EEG recording for approximately 10 minutes. This was performed to investigate how nervous tissue within a living subject reacts to the ultrasound as compared to dead nervous tissue.

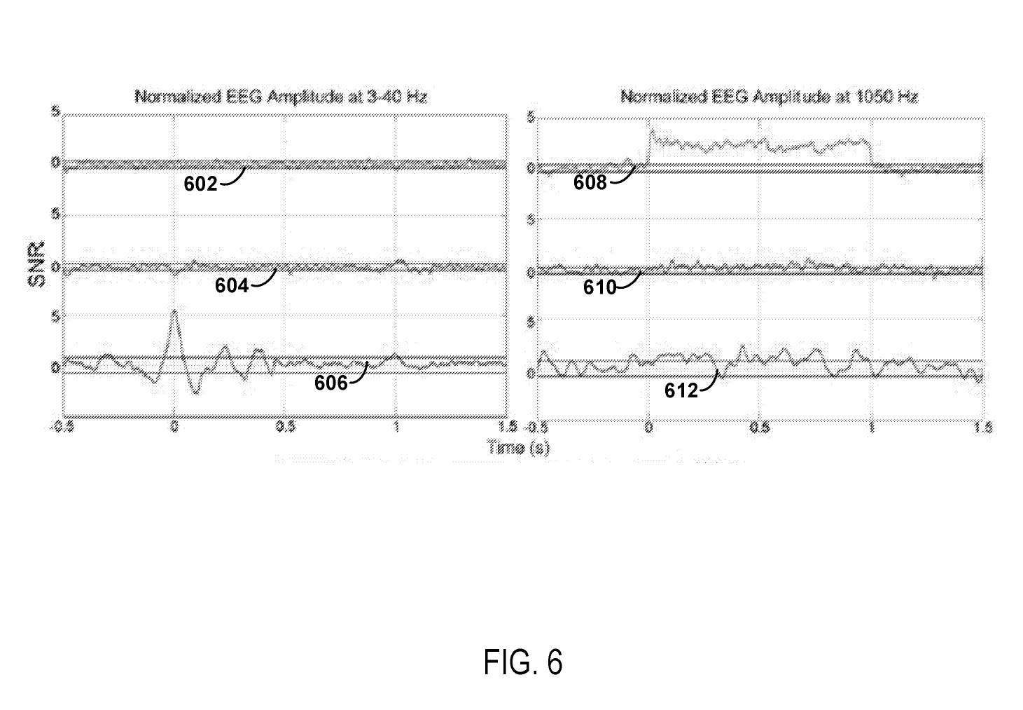

FIG. 6 depicts EEG amplitude response with respect to differing target characteristics. Curves 602, 604, and 606 depict grand average evoked potentials (EP) in the 3-40 Hz band (e.g., representing natural neural activity). The curves 602, 604, and 606 correspond respectively to living rat tissue, dead rat tissue, and alginate over the course of the two second pFU off/on trial described above with reference to FIG. 5. Curves 608, 610, and 612 depict grand average EP at 1050 Hz (e.g., representing neural activity partially induced by the applied ultrasound). The curves 608, 610, and 612 correspond respectively to living rat tissue, dead rat tissue, and alginate over the course of the two second pFU off/on trial. The time course shown is from 0.5 seconds prior to pFU stimulation to 1.5 seconds after pFU stimulation, to allow illustration of pFU-stimulation onset and offset effects. The data is shown units of signal-to-noise ratio relative to the “pFU-off” period. Black lines for each graph indicate the 99.5% confidence intervals. Note that the EP are shown as an SNR of measured voltage averaged over 3-40 Hz, while the 1050 Hz response is shown as the SNR of the amplitude of the band pass filtered voltage between 1040 and 1060 Hz. A comparison of curves 608 and 610 show that nervous tissue within a living subject reacts more strongly to the applied ultrasound than dead nervous tissue.

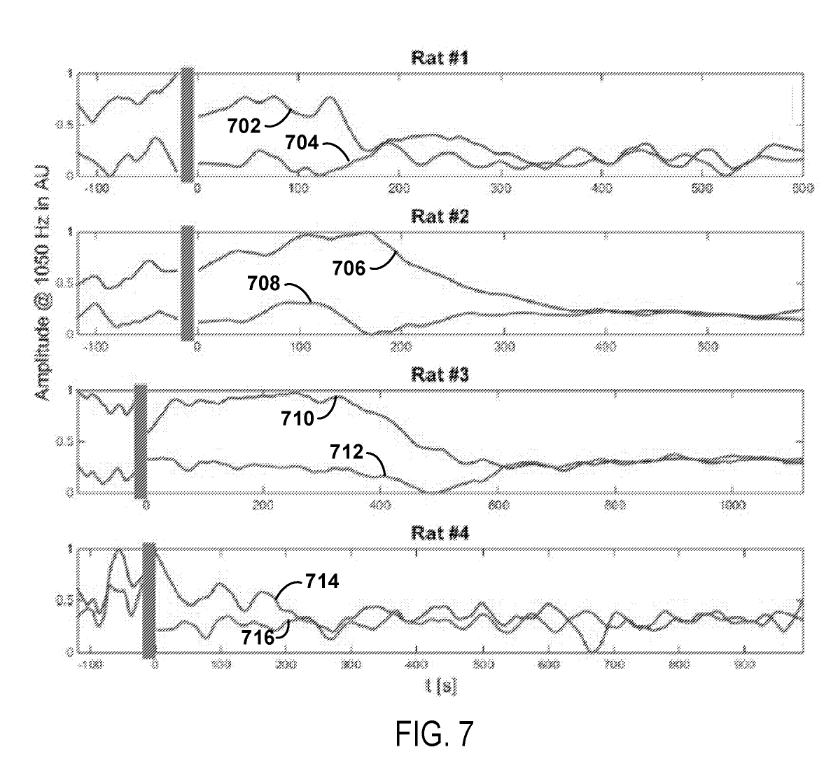

FIG. 7 depicts EEG amplitude response of four rat subjects with respect to time. Curves 702, 706, 710, and 714 depict derived responses at 1050 Hz during the one-second pFU-on period. The curves 704, 708, 712, and 716 depict the corresponding EEG-derived 1050 Hz amplitude during the one-second pFU-off period. The curves 702-716 are scaled to their common maximum value and have been smoothed with a moving average filter of one minute in duration. The noise floor measured at 900 Hz has also been subtracted from each curve 702-716. The x-axis shows the time in seconds. The time before zero indicates time before injection of Beuthanasia, that is, the time the rat is under anesthesia but otherwise has an “active” brain. The time after zero indicates the time after injection of Beuthanasia, that is, the time the rat is dead (or dying) and has an “inactive” brain state.

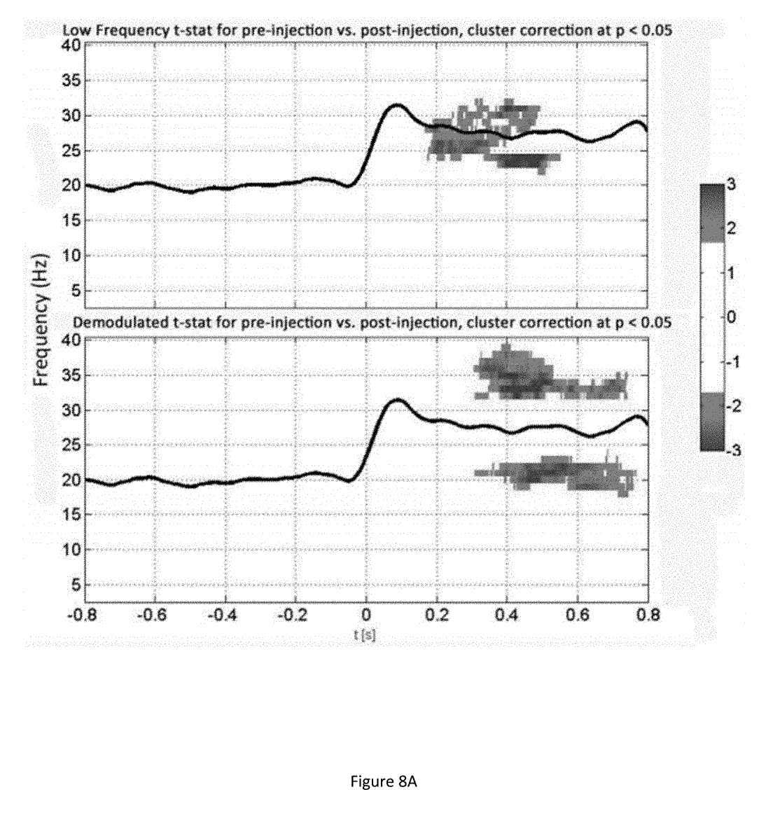

FIG. 8 depicts EEG frequency response with respect to differing target characteristics. Both graphs depict grand average evoked potentials of the normalized ratio spectrum between pFU-on and pFU-off conditions for all sensor channels from all four rats. The spectrum for the pre-injection state is shown on the left, and the post injection state is on the right. The X-axis is a log-scale of frequencies, ranging from 5 Hz to 2000 Hz. The SNR value at the stimulation frequency in both states is annotated and shown with the frequency-specific 99.5% confidence interval (small black lines around the 1050 Hz peak). Otherwise the black lines indicate the confidence interval across all frequencies. The peaks annotated with (A) and (B) are located at, respectively, 2100 Hz (1st harmonic of 1050 Hz) and 1650 Hz (a wrap-around of 3150 Hz, i.e., a 3rd harmonic of 1050 Hz, which occurs due to limited the sampling frequency of 4800 Hz). A comparison of the graphs on the left and the right show that living tissue reacts more strongly to the applied ultrasound than dead tissue.

While various example aspects and example embodiments have been disclosed herein, other aspects and embodiments will be apparent to those skilled in the art. The various example aspects and example embodiments disclosed herein are for purposes of illustration and are not intended to be limiting, with the true scope and spirit being indicated by the following claims.

1. A method comprising:
applying ultrasound waves to a particular portion of a nervous tissue, wherein the ultrasound waves are pulsed at a pulse repetition frequency;
detecting an electrical signal originating from at least the particular portion of the nervous tissue;
extracting a component of the electrical signal that oscillates at an oscillation frequency that is equal to (a) the pulse repetition frequency, (b) a subharmonic frequency of the pulse repetition frequency, or (c) a harmonic frequency of the pulse repetition frequency; and
processing the extracted component to obtain one or more components that oscillate at respective frequencies that are unequal to the pulse repetition frequency.

2. The method of claim 1, wherein the nervous tissue is nervous tissue within a living subject.

3. The method of claim 1, wherein the pulse repetition frequency is greater than 1 Hz and less than 20 MHz.

4-5. (canceled)

6. The method of claim 1, wherein the ultrasound waves have a carrier frequency that is greater than 20 kHz and less than 200 MHz.

7-9. (canceled)

10. The method of claim 1, wherein the ultrasound waves have an IsPTA within a range of 0.01-20 W/cm² as measured within the nervous tissue.

11. (canceled)

12. The method of claim 1, wherein the nervous tissue comprises central nervous system tissue.

13-14. (canceled)

15. The method of claim 1, wherein applying ultrasound waves comprises applying ultrasound waves via an ultrasound transducer that is positioned external to the nervous tissue.

16. The method of claim 1, wherein ultrasound waves comprise ultrasound waves that are focused upon the particular portion of the nervous tissue.

17-18. (canceled)

19. The method of claim 1, wherein detecting the electrical signal comprises detecting the electrical signal via an electrode that is attached external to the nervous tissue.

20-25. (canceled)

26. The method of claim 1, wherein processing the extracted component comprises demodulating the extracted component.

27-29. (canceled)

30. The method of claim 1, wherein the one or more components originate from the particular portion of the nervous tissue.

31-34. (canceled)

35. The method of claim 1, wherein the respective frequencies at which the one or more obtained components oscillate are greater than 3 Hz and less than 1000 Hz.

36. (canceled)

37. The method of claim 1, further comprising using the one or more obtained components to monitor nervous tissue activity within a living subject.

38-41. (canceled)

42. The method of claim 37, wherein the subject has or exhibits symptoms of a nervous system disorder, further comprising using the one or more obtained components to monitor or guide treatment of the nervous system disorder.

43. The method of claim 42, wherein using the one or more obtained components to monitor treatment of the disorder comprises using the one or more obtained components to monitor progress of ultrasound therapy.

44. (canceled)

45. The method of claim 1, further comprising:
using the one or more obtained components to determine one or more parameters for applying therapeutic ultrasound to the nervous tissue within a living subject; and
altering neurological activity of the nervous tissue by applying the therapeutic ultrasound to the nervous tissue within the living subject according to the determined one or more parameters.

46-48. (canceled)

49. The method of claim 45, wherein altering the neurological activity comprises stimulating a first portion of the nervous tissue such that the first portion of the nervous tissue functions to alter neurological activity of a second portion of the nervous tissue

50-53. (canceled)

54. A system comprising:
one or more processors;
an ultrasound transducer;
one or more sensors; and
a computer-readable medium storing instructions that, when executed by the one or more processors, cause the monitoring system to perform the method of:
applying ultrasound waves to a particular portion of a nervous tissue, wherein the ultrasound waves are pulsed at a pulse repetition frequency;
detecting an electrical signal originating from at least the particular portion of the nervous tissue:
extracting a component of the electrical signal that oscillates at an oscillation frequency that is equal to (a) the pulse repetition frequency, (b) a subharmonic frequency of the pulse repetition frequency, or (c) a harmonic frequency of the pulse repetition frequency; and
processing the extracted component to obtain one or more components that oscillate at respective frequencies that are unequal to the pulse repetition frequency.
